(19) United States
(12) Patent Application Publication
(10) Pub. No.: US 2008/0312742 A1
(43) Pub. Date: Dec. 18, 2008

(54) Anterior Spinal Fusion and Fixation Cage with Integrated Plate and Method of Use

(76) Inventor: Dennis Lee Abernathie, Columbia, MO (US)

Correspondence Address: KIMSEY PATENT SERVICES, LLC, 804 SHAMROCK AVENUE, BOONVILLE, MO, 65233, US

(21) Appl. No.: 11/762,046

(22) Filed: Jun. 12, 2007

(51) Int. Cl. A61F 2/44; A61B 17/58; A61B 17/04

(52) U.S. Cl. 623/17.16; 623/17.11; 606/301

(57) ABSTRACT

The present invention provides a fixation and fusion cage with interlocking plate comprising a central cage portion and interlocking plate that rigidly attaches to the front of the cage. The cage can be varied in shape to account for various clinical necessities such as lordosis. The invention may be installed in an offset configuration so as to avoid damage to blood vessels. The device may be constructed from radiolucent material to allow for visualization of bone fusion within the cage.

### BACKGROUND OF THE INVENTION

The present invention relates generally to an implantable device for promoting the fixation and fusion of adjacent bony structures, and a method of using the same. More specifically, the present invention relates to an anterior fixation and fusion cage with an optional integrated plate that may be inserted into an intervertebral space, and a method of using the same.

Fusion cages provide a space for inserting bone graft between adjacent portions of bone. These cages are used to increase disc height and provide stability to damaged or degenerative spines. Such cages are made from a variety of materials. These materials include but are not limited to titanium, carbon fiber, and polyetheretherketone (PEEK). Fusion cages are often hollow and can be porous to allow for maximum bone growth through the cage and into adjacent vertebral bodies. Cages of the nature described here are used to treat numerous spinal disorders including but not limited to degenerative disc disease, degenerative facet joint disease, isthmic and degenerative spondylolisthesis, and spinal stenosis.

Fixation cages are used to stabilize spinal motion segments, and to reduce relative motion between adjacent levels. Such cages provide rigid support for spinal structures either before or in place of spinal fusion. Similar to fusion cages, fixation cages are made from a variety of materials. These materials include but are not limited to titanium, carbon fiber, and polyetheretherketone (PEEK).

Many different surgical approaches are used to implant fixation and fusion cages. These approaches include anterior lumbar interbody fusion (ALIF), posterior lumbar interbody fusion (PLIF), transforaminal interbody fusion (TLIF), and extreme lateral interbody fusion (XLIF). The cages are generally inserted through a traditional open incision; however, laparoscopic, percutaneous, or other minimally invasive techniques may also be used.

Regardless of the approach, the typical procedure for inserting a common spinal fixation or fusion cage is the same. First, the site is exposed either through an open or minimally invasive exposure. Next, the intervertebral disc is removed. The endplates of each vertebrae are prepared so that they will accept the cage and to provide an environment conducive to osseous growth. The process of preparing the endplates also generally includes determining the proper size of implant to ensure adequate distraction of the construct. The size and orientation of the cage is chosen to ensure proper lordosis and stability of the postoperative spine.

The ALIF approach is common and well established; however, this procedure suffers from disadvantages due to current implant design. This procedure is performed in close proximity to the large blood vessels that supply the legs, thereby risking damage to these blood vessels, which can result in excessive blood loss. This proximity to blood vessels often results in the anterior fusion cage being placed lateral to midline in an orientation that may exceed that which the implant was designed to accommodate. Anterior cages also are at risk for further migration into the disc space potentially causing complications.

The surface area of contact between traditional interbody devices and adjacent vertebrae is less than the area of contact for an intact disc. This surface area reduction can lead to stress-induced failure such as subsidence or abnormal bone remodeling. This problem is often compounded by implants that have sharp corners generating stress concentrations that accelerate failure. The more anatomical agreement between an implant and the prepared surface of the vertebrae the better the chance for a successful fusion. Also, the larger the surface area in contact the more distributed the loading will be, reducing the chance for subsidence.

A problem with existing titanium cages is that it is difficult to assess spinal fusions postoperatively because the metal of the cage interferes with attempts to evaluate the fusion by x-ray. Radiolucent cages, such as those made from either carbon fiber or PEEK, have been used to provide better postoperative visualization of spinal fusions.

Anterior fusion cages are many times used in conjunction with some form of anterior plate. In general the purpose of the fusion cage is to resist axial compression, and the purpose of a plate is to resist tensile forces. Traditionally anterior cages and plates are separate from one another, and are not designed to interact in distributing loads. This configuration requires surgical time to first install the cage and then install the plate. The patient and surgeon would both benefit from a reduction in the surgical time required to install the anterior cage and plate.

Anterior plates are beneficial for increasing the stability of the surgical construct until sufficient fusion has occurred. Traditional plates are designed independent of the cage with which they will be used. Therefore there are no beneficial interactions between the plate and cage. Traditional plates suffer from large profiles. Plates such as these are installed very near the blood vessels that supply the lower extremities. Plates with large profiles run the risk of irritating or rupturing these blood vessels. Traditional cages are designed to be installed at or very near the midline. This can be difficult to accomplish surgically due to patient anatomy.

What is needed then is a spinal fixation and fusion cage suitable for an ALIF procedure that integrates with a plate to prevent migration, provides optimum anatomical agreement between the cage and adjacent vertebrae, can be viewed postoperatively using x-ray, can be used in conjunction with a complementary plate to help reduce operating time, and is able to be installed off midline to avoid disruption of blood flow to the lower extremities.

### BRIEF SUMMARY OF THE INVENTION

The present invention provides a fixation and fusion cage having an upper contoured surface, a lower contoured surface, a first side wall, and a second side wall, the upper and lower contoured surfaces and first and second side walls forming opposing upper and lower surfaces and opposing side walls, respectively, of the cage. In one aspect of the present invention, at least one of the contoured surfaces or side walls is sized and shaped to form an interlocking portion for attachment of a plate portion thereto. In another aspect of the present invention, an interlocking plate portion is provided, the interlocking plate portion being adapted to engage the interlocking portion of the cage such that the plate portion is rigidly attached to the cage. The interlocking plate portion is preferably shaped such that, depending on the orientation of the interlocking plate when attached to the present cage, the present cage is either positioned along the midline of the vertebral column when inserted into a vertebral space, or is offset with respect to the midline. The upper and lower surfaces preferably include a contour shaped to mate with an adjacent vertebral shape when the present fusion cage is inserted into an intervertebral space.

The interlocking plate of the present fixation and fusion cage preferably has a central opening formed therein. Likewise, each of the upper and lower surfaces and first and second side walls preferably have at least one opening formed therein.

The present fixation and fusion cage is preferably constructed from at least one radiolucent compound, though various compounds may be used in the construction of the cage including, but not limited to, titanium, bone, carbon fiber, polyetheretherketone, nylon, polycarbonate, stainless steel, chromium-cobalt, or combinations thereof.

In another aspect, the present invention provides a screw for attaching a plate associated with the present fixation and fusion cage to a bony portion of a patient's anatomy, such as a vertebrae. The screw preferably includes a bullet tip, a first set of threads sized and shaped to engage the interior wall of a bore in said bony portion, and a second set of threads sized and shaped to engage an opening in said plate. The screw further preferably includes a tool-receiving portion for accepting a tool therein for the purpose of tightening or loosening the screw.

### BRIEF DESCRIPTION OF THE DRAWINGS

FIG. 1 is a perspective view of a fusion and fixation cage with interlocking plate constructed in accordance with the teachings of the present invention.

FIG. 2 is an exploded view of a fixation and fusion cage with interlocking plate constructed in accordance with the teachings of the present invention, showing the interlocking plate separate from the fixation and fusion cage.

FIG. 3 is a lateral view of an interlocking plate constructed in accordance with the teachings of the present invention.

FIG. 4 is a top view of an interlocking plate constructed in accordance with the teachings of the present invention.

FIG. 5 is a front view of an interlocking plate constructed in accordance with the teachings of the present invention.

FIG. 6 is a perspective view of one embodiment of the present invention showing one possible orientation of attachment of the interlocking plate to the front of the cage, and positioned in front of an opening between adjacent vertebrae.

FIG. 7 is a perspective view of one embodiment of the present invention showing the interlocking plate attached to the front of the cage, and one possible arrangement for insertion into an opening between adjacent vertebrae.

FIG. 8 is a front view of one embodiment of the present invention showing the cage and interlocking plate in position in an opening between adjacent vertebrae.

FIG. 9 is a side view of one embodiment of the present invention showing the cage and interlocking plate in position in an opening between adjacent vertebrae.

FIG. 10 is a perspective view of a fusion and fixation cage with interlocking plate constructed in accordance with the teachings of the present invention, wherein the interlocking plate is offset relative to the fusion cage.

FIG. 11 is a top view of a fusion and fixation cage with interlocking plate constructed in accordance with the teachings of the present invention, wherein the interlocking plate is offset relative to the fusion cage.

FIG. 12 is a perspective view of a fusion cage constructed in accordance with the teachings of the present invention, said fusion cage having posts adapted to engage recessed portions of an interlocking plate such that the plate is offset relative to the cage.

FIG. 13 is a perspective view of a screw for attaching the present fusion cage and interlocking plate to the bone of a patient.

FIG. 14 is a perspective view of one alternative embodiment of an interlocking plate constructed in accordance with the teachings of the present invention.

### DETAILED DESCRIPTION OF THE INVENTION

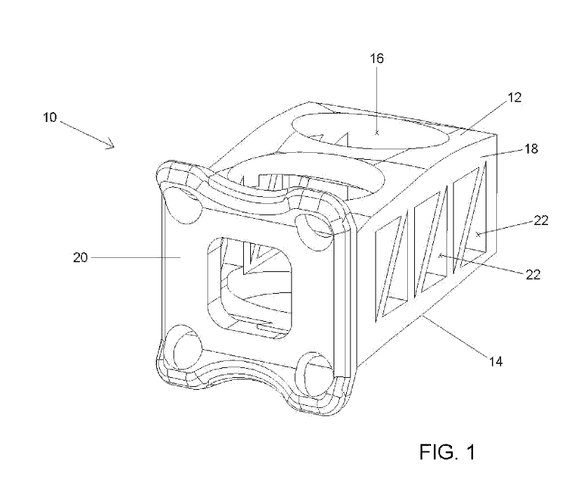

Several embodiments of a fixation and fusion cage with interlocking plate constructed in accordance with the teachings of the present invention are shown in the figures, wherein like numerals indicate like parts. Turning now to FIG. 1, a fixation and fusion cage constructed in accordance with the teachings of the present invention is indicated generally by the numeral 10. Fixation and fusion cage 10 of the present invention is preferably generally rectangular in shape and includes an upper cage surface 12 and a lower cage surface 14 (hereinafter referred to as upper cage surface and lower cage surface). Each of the upper and lower cage surfaces is sized and shaped to form a contoured surface that agrees with the anatomy of the contacting vertebral surfaces between which the present cage will be used. Each of upper and lower cage surfaces 12 and 14 also preferably has a plurality of openings 16 formed therein. Upper cage surface 12 and lower cage surface 14 are supported by side portions 18 and 19 (not shown), which extend between upper and lower cage surfaces 12 and 14 and form opposing sidewalls thereof. Upper and lower surfaces 12 and 14, along with side portions 18 and 19, define an interior space of fixation and fusion cage 10. Side portions 18 and 19 preferably have a plurality of openings, 22 and 23 respectively, therein. Numeral 20 identifies an interlocking plate adapted for use with the fixation and fusion cage of the present invention. As will become apparent below, interlocking plate 20 is preferably rigidly attached to cage 10.

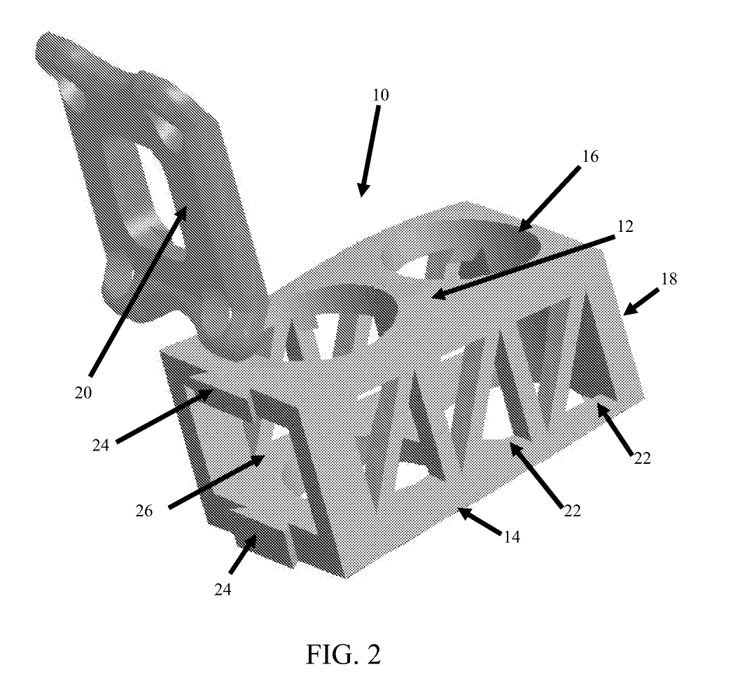

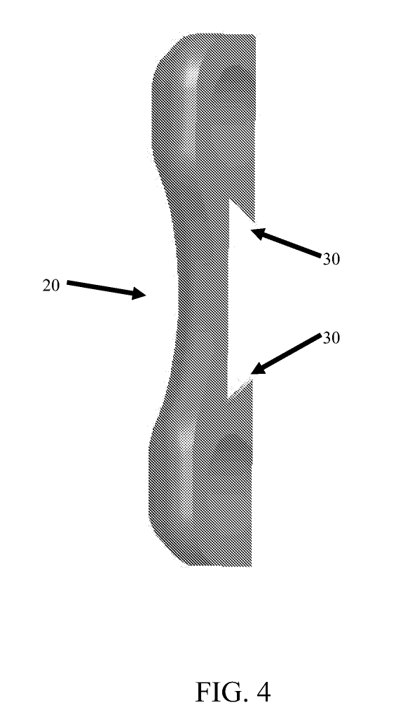

FIG. 2 is an exploded view of a fixation and fusion cage 10 and interlocking plate 20 constructed in accordance with the teachings of the present invention. The embodiment of interlocking plate 20 shown in FIG. 2 further includes interlocking posts 24 present on a side edge of each of side portions 18 and 19. As will be described below, interlocking posts 24 are sized and shaped to fit into corresponding recesses 30 (as shown in FIG. 4) of interlocking plate 20. In FIG. 2, a large central opening 26 of cage 10 is also shown. While two interlocking posts 24 are shown in the figure, it is contemplated that only one interlocking post 24 may be used or, alternatively, that more than two interlocking posts 24 may be used, the interlocking posts 24 located along any of the side edges of upper or lower surfaces 12 and 14 or side portions 18 and 19. Further, while the structures of interlocking posts 24 and corresponding recesses 30 shown in the figures are preferred, any suitable structure for rigidly attaching interlocking plate 20 to fusion cage 10 may be used. Interlocking posts 24, for example, may be located on interlocking plate 20, while recesses 30 are located within side edges of any of upper and lower surfaces 12 and 14 and/or side portions 18 and 19. Additionally, the interlocking portion may be a separate portion from the upper or lower surfaces or side portions, and may be fixedly attached thereto. Although interlocking posts and recessed portions are referred to individually, they may be collectively referred to throughout this specification, and in the claims, as ‘interlocking portions.’

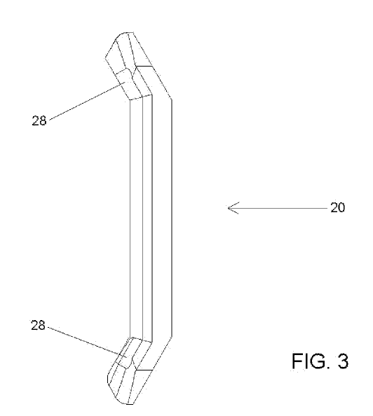

FIG. 3 is a side view of one embodiment of interlocking plate 20. As shown in the figure, interlocking plate 20 includes two angled portions 28 that allow interlocking plate 20 to be recessed into a disk space between adjacent vertebrae. The angle of incline of angled portions 28 relative to the rest of interlocking plate 20 may vary depending on the specific use of the present device, and many suitable angles will be apparent to those of skill in the art upon reading this disclosure. A general angle may be determined by measuring the appropriate corresponding angle in a group of patients and then mathematically extracting a ‘normal’ result. Alternatively, the present device may be customized to fit a particular patient, in which case the angle provided will match the needs of that patient's anatomy.

FIG. 4 shows a top view of one embodiment of interlocking plate 20. In this embodiment of interlocking plate 20, interlocking recesses 30 are used to create a rigid attachment of the plate to interlocking posts 24 of the fixation and fusion cage 10. Any suitable mechanism for rigidly affixing interlocking plate 20 to fusion cage 10 may be used, and different locking mechanisms will result in different shapes or configurations of interlocking plate 20 and fusion cage 10. Many such configurations will be readily apparent to those of skill in the art upon reading this disclosure, and it is contemplated that such variations remain well within the spirit and scope of the present invention.

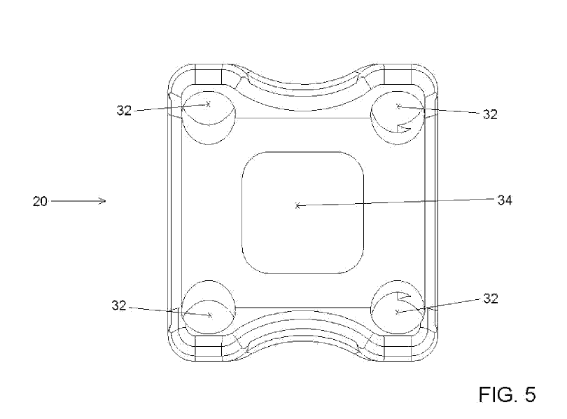

FIG. 5 is a front view of one embodiment of interlocking plate 20. Screw holes 32 are preferably provided so that screws (such as screw 150, described below) may be used to rigidly attach interlocking plate 20 to the adjacent vertebrae. In an alternative embodiment of the present invention, the screw holes are designed to allow motion between the screw and plate, thereby allowing non-rigid fixation of the plate to the adjacent vertebrae. Further, in the embodiment of the present invention shown in FIG. 5, a central opening 34 is included in interlocking plate 20. It is contemplated, however, that central opening 34 can be provided in different sizes and shapes other than that shown in the figure, or that a plurality of openings may instead be used. In still another embodiment of the present invention, central opening 34 is eliminated, thereby creating an interlocking plate with no central opening. Further, although screw holes 32 are described herein as 'screw holes' it is contemplated that any suitable fastener may be used in order to fasten interlocking plate 20 to a vertebrae of a patient.

Although fixation and fusion cage 10 is shown in the figures being used in conjunction with interlocking plate 20, it is contemplated that either cage 10 and/or interlocking plate 20 can be used separately from one another.

Fixation and fusion cage 10 and interlocking plate 20 are preferably composed of a radiolucent material such as, for example, polyetheretherketone (PEEK) or carbon fiber. Other suitable materials may also be used. Examples of suitable materials include, but are not limited to, various plastics, such as, for example, nylons, polycarbonates, and polyketones. Suitable metallic substitutes may include, but are not limited to, titanium, stainless steels, and chromium-cobalt.

Although the figures show fixation and fusion cage 10 having two openings 16 in the upper and lower cage surfaces 12 and 14, it is contemplated that any number of openings of varying size and shape may be used. For example, a plurality of smaller openings or a single larger opening may be used. Similarly, it is contemplated that openings 22 and 23 in side portions 18 and 19 may be comprised of various other sizes and shapes.

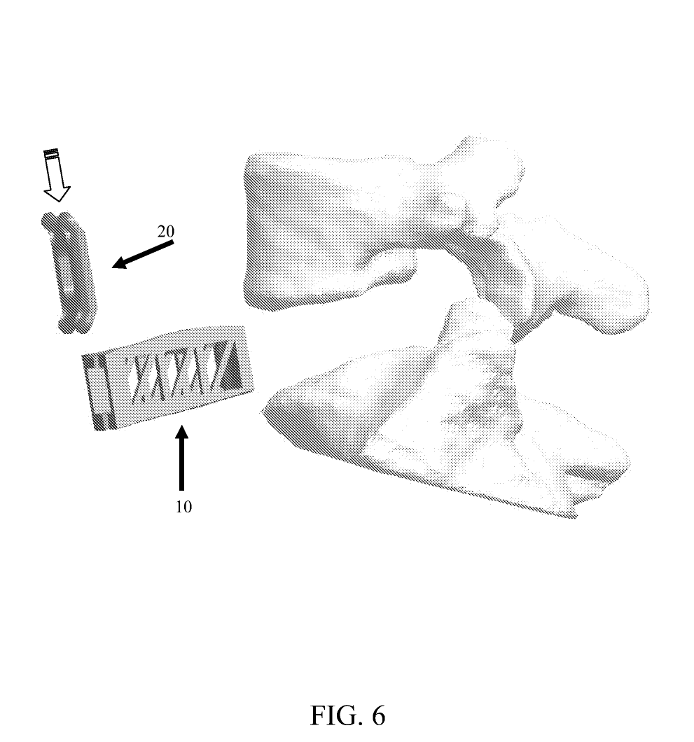

FIG. 6 shows one embodiment of the assembly process of a fixation and fusion cage 10 and the interlocking plate 20 of the present invention. In this embodiment, recesses 30 (not shown, but located in an inner surface of interlocking plate 20 as shown in previous figures) of interlocking plate 20 engage posts 24 of fixation and fusion cage 10 either from the top or bottom. Posts 24 engage recesses 30 slidingly, by positioning posts 24 and recesses 30 adjacent one another from either the top or bottom, and then sliding interlocking plate 20 either in either an upward or downward manner, as appropriate, such that interlocking posts 24 are slidingly received within recesses 30, thereby engaging interlocking plate 20 with fusion cage 10.

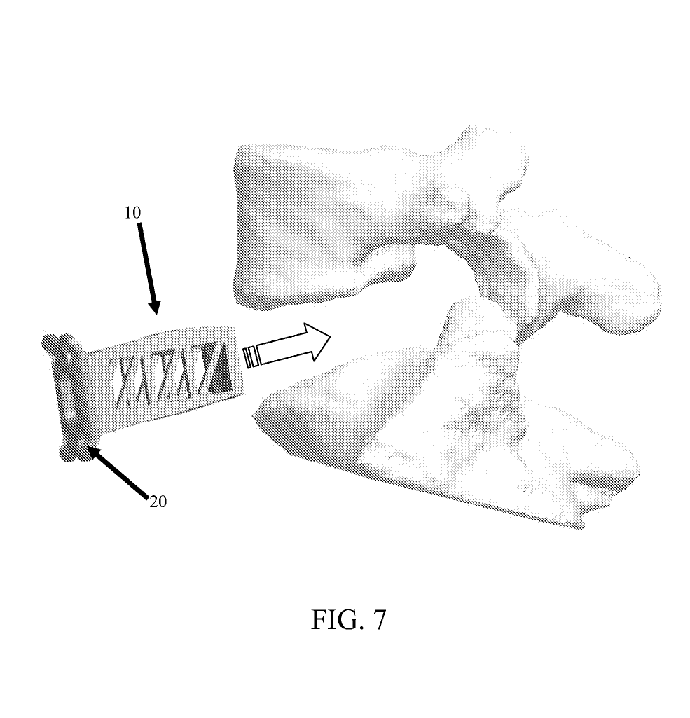

Once interlocking plate 20 is locked in place by engaging with recesses 30 of fusion cage 10, the entire assembly is inserted from the front (in terms of the anatomical orientation of the patient) into a space between adjacent vertebrae. One embodiment of this installation is shown in FIG. 7. It is contemplated that an installation tool may be used to drive the assembled cage into the disc space. During procedures in which such a tool is used, it is contemplated that this tool preferably fits into opening 34 of interlocking plate 20 in such a manner as to allow a user of the tool to push the assembled cage into place.

In one embodiment of the present invention, interlocking plate 20 is secured to adjacent vertebrae using bone screws (not shown) of typical design passing through screw holes 32. In one embodiment of interlocking plate 20, the bone screws will lock to interlocking plate 20 in the final installed configuration. In another embodiment the screws will not lock to interlocking plate 20, allowing for movement between the screws and interlocking plate 20. In an alternative embodiment of the present invention, the interlocking plate of the present device is adapted for use with specialized bone screws 150 (described below).

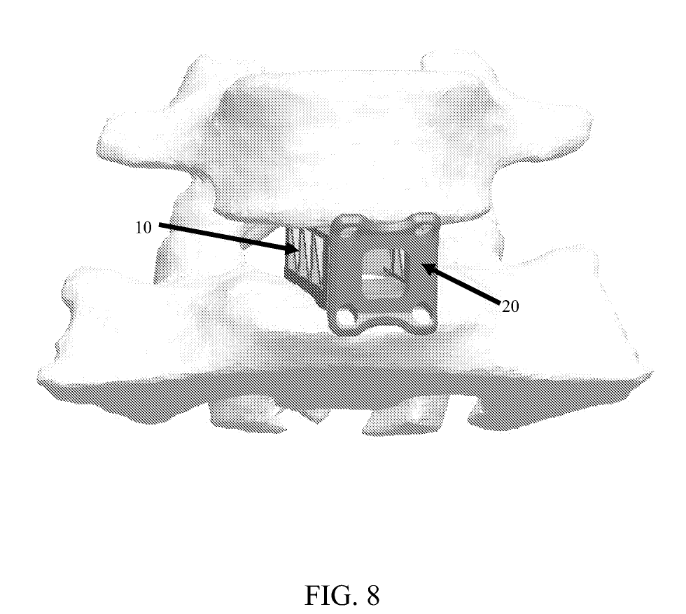

FIG. 8 shows one embodiment of an installed configuration of the present fusion cage 10 and interlocking plate 20. In this embodiment of the invention the assembled cage 10 and interlocking plate 20 are positioned such that they are offset from the midline of the vertebral column. This allows for the present device to be installed adjacent to blood vessels running along the anterior aspect of the vertebral column. In one embodiment of the present invention, interlocking plate 20 is shaped such that a 180 degree rotation of interlocking plate 20 from its usual orientation (as shown, for example, in FIG. 2) results in the assembled cage 10 and interlocking plate 20 being offset from the midline of the vertebral column, whereas utilizing interlocking plate 20 in its usual orientation results in cage 10 and interlocking plate 20 being centered along the midline of the vertebral column.

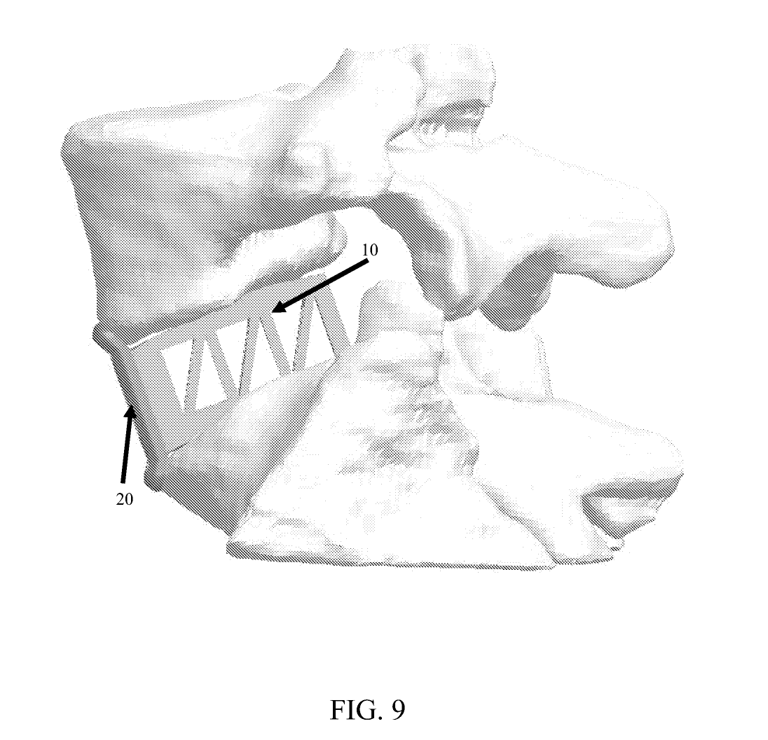

FIG. 9 is a lateral view of one embodiment of the assembled construct. In this figure, it can be seen that upper and lower cage surfaces 12 and 14 mate with the contour of the vertebral endplates between which cage 10 is being inserted. One method of determining the contours of the intervertebral space includes digitizing the bone structure of the vertebral endplates, then analyzing the void between the vertebral endplates across multiple samples (patients) to obtain a general contour for the vertebral endplate. This method for modeling the contours of an anatomical space is described in more detail in U.S. patent application Ser. No. 11/551,591, by Abernathie, entitled “Improved Spinal Fusion Cage, Method of Design, and Method of Use,” which is incorporated herein by reference. It is contemplated, however, that any suitable method of determining the contour of the vertebral endplates (and therefore the corresponding contour of upper and lower cage surfaces 12 and 14) may be used without departing from the spirit or scope of the present invention.

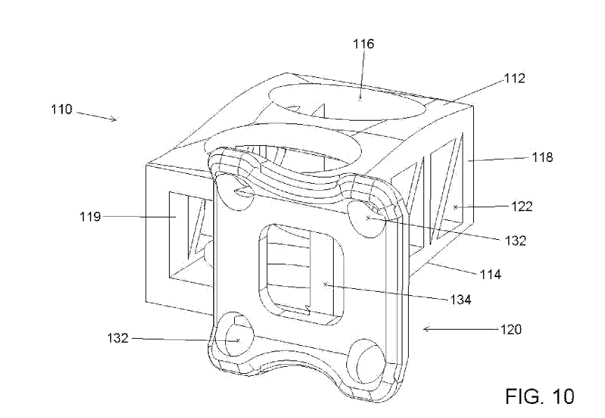

FIG. 10 provides a perspective view of an alternative embodiment of the present fixation and fusion cage having an interlocking plate associated therewith. Fusion cage 110 in FIG. 10 includes upper and lower contoured cage surfaces 112 and 114, and preferably also includes a plurality of openings 116 formed therein. Side portions 118 and 119 also preferably include a plurality of openings 122. These aspects of fusion cage 110 are substantially similar to those equivalent portions of fusion cage 10, described above. Likewise, interlocking plate 120 preferably includes portions substantially similar to those described with respect to interlocking plate 20, above. For example, interlocking plate 120 includes screw holes 132 and central opening 134, and unless otherwise stated these aspects of interlocking plate 120 are substantially similar to the corresponding aspects of interlocking plate 20.

Fusion cage 110 an interlocking plate 120 differ from the embodiment of the present invention described above (i.e. fusion cage 10 and interlocking plate 20) primarily in that interlocking plate 120 is adapted to attach to fusion cage 110 (and fusion cage 110 is adapted to receive interlocking plate 120) such that interlocking plate 120 is offset with respect to a centerline along the longitudinal axis of fusion cage 110. Thus, while interlocking plate 120 is offset with respect to the vertebral column of a patient in whom the present device has been implanted, for the reasons described above, fusion cage 110 is properly positioned and centered.

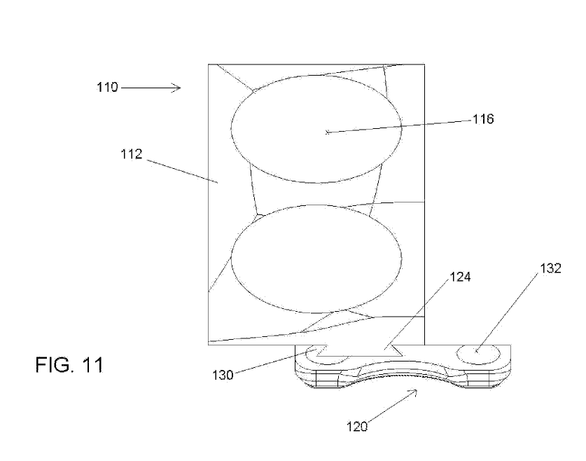

FIG. 11 provides a top view of the embodiment of fusion cage 110 and interlocking plate 120 shown in FIG. 10. In this drawing, interlocking post 124 of fusion cage 110 is clearly shown, and is seen to be located such that interlocking plate 120 attaches to interlocking post 124 of fusion cage 110 in an offset position relative to a centerline along the longitudinal axis of fusion cage 110. As is also shown, interlocking plate 120 has a recessed portion 130 adapted to receive interlocking post 124 of fusion cage 110. Although interlocking post 124 and recessed portion 130 are shown in the drawings, it is contemplated that any suitable member of attaching interlocking plate 120 to fusion cage 110 may be used, including reversing interlocking post 124 and recessed portion 130 such that recessed portion 130 is located along a surface of fusion cage 110 and interlocking post 124 is located along a surface of interlocking plate 120.

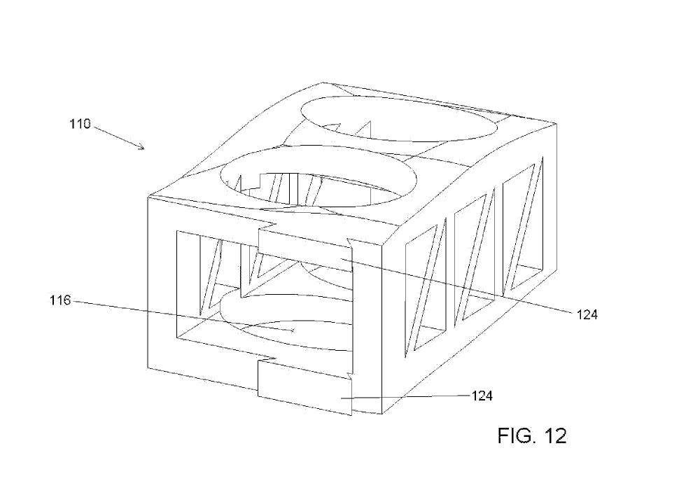

FIG. 12 provides a perspective view of fusion cage 110 without interlocking plate 120 attached thereto, such that interlocking posts 124 of fusion cage 110 are visible. As shown in the figure, the present invention preferably includes two interlocking posts 124. Interlocking plate 120 preferably includes two recessed portions 130 adapted to receive interlocking posts 124 of fusion cage 110. It is contemplated that fusion cage may be provided with only one interlocking post 124, and that interlocking plate 120 include only one recessed portion, or that more than two of interlocking posts 124 and recessed portions 130 may also be utilized. As noted previously, in each of the various embodiments of the present invention and suitable method of affixing interlocking plate 120 to fusion cage 110 may be used without departing from the spirit or scope of the present invention.

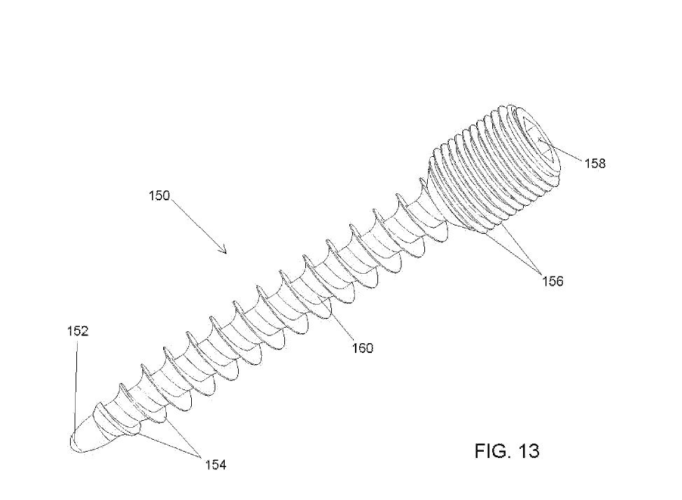

FIG. 13 provides a perspective view of one embodiment of a screw 150 adapted for use in attaching an interlocking plate of the present invention to a vertebrae or other bony surface of a patient in whom the present device is implanted. Screw 150 includes a number of features that render it particularly useful for attachment of a fusion cage and interlocking plate such as that described above. Screw 150 includes a bullet tip 152, which is a small extension of the screw root 160 that extends longitudinally to form an area of screw 150 before first threads 154 begin. Bullet tip 152 is preferably rounded and allows screw 150 to be placed into a hole in a vertebrae or other bony surface of a patient's anatomy such that screw 150 can be properly aligned prior to being inserted into place. First threads 154 are preferably self-tapping, such that screw 150 creates its own thread when being inserted into a vertebrae or other bony structure of a patient. First threads 154 extend along screw root 160, which is substantially uniform in diameter, until giving way to second threads 156 of screw 150. The outer diameter of second threads 156 is preferably greater than that of first threads 154, and the distance between second threads 156 is preferably less than the distance between threads 154. The relative outer diameter and distance between the two sets of threads differs based on the different functions of these two portions of screw 150. First threads 154 are adapted to pass through screw holes 32 of interlocking plate 20 and into a bony structure of a patient in whom the present device is being implanted. Second threads 156 are adapted to engage an inner wall of screw holes 32, with the outer perimeter of screw holes 32 being received within the spaces between second threads 156, thereby securing screw 150 to interlocking plate 20.

At an end of screw 150 opposite bullet tip 152, screw 150 preferably includes a shaped opening 158 adapted to receive a tool for turning, and thereby tightening or loosening, screw 150. Any suitable tool may be used for this task, and shaped opening 158 may be provided in any suitable size, shape, or depth necessary to accommodate the tool to which screw 150 is adapted. Shaped opening 158 may, for example, be sized and shaped consistent with various well-known screw heads, such as for example slotted screw heads, phillips screw heads, hex screw heads, square screw heads, and the like.

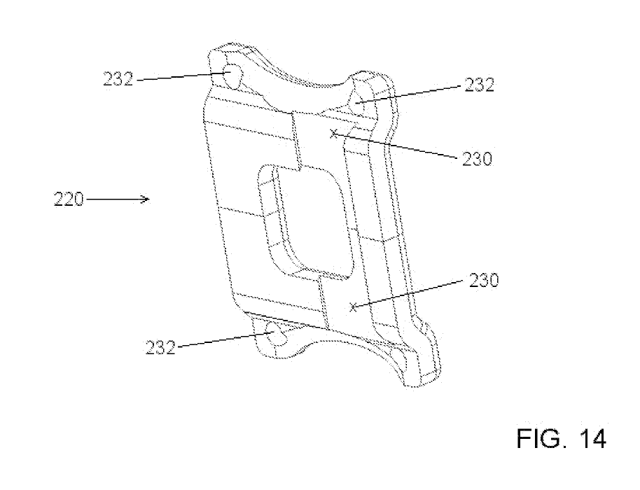

FIG. 14 provides a perspective view of one alternative embodiment of an interlocking plate constructed in accordance with the teachings of the present invention. With reference to the embodiments of the interlocking plate of the present invention described previously, it is noted that the screws used in conjunction with those plates to attach the plates to the vertebrae enter the vertebrae at approximately a forty-five degree angle. This can be seen, for example, in the configuration of screw holes 32 shown in FIG. 5 and described above. In the embodiment of interlocking plate 220 shown in FIG. 14, screw holes 232 are positioned such that any screws inserted into those screw holes will enter the vertebrae at approximately a ninety-degree angle. In some instances, this can provide easier access to the screw heads than in embodiments of the present invention wherein the screws enter the vertebrae at closer to a forty-five degree angle. It is contemplated, however, that any suitable angle of screw hole, and any suitable angle of screw entering the vertebrae, may be used without departing from the spirit or scope of the present invention. In other respects, interlocking plate 220 functions substantially similar to the embodiments of the interlocking plate described above. For example, recessed portions 230 are present, whereby the interlocking plate may be associated with a cage. As with other embodiments of the interlocking plate described above, the plate may be inverted (rotated one-hundred eighty degrees) in order to change the offset of the plate relative to the cage.

In another alternative embodiment of the present invention, it is contemplated that an interlocking plate is provided having a first edge that is angled, such as an edge of interlocking plate 20, shown in FIG. 5, such that the screw holes associated therewith are angled at such that screws inserted therethrough enter a vertebrae at approximately a forty-five degree angle, while a second edge of the plate is similar to an edge of interlocking plate 220, shown in FIG. 14, such that screws inserted through the screw holes along that edge of the plate enter the vertebrae at approximately a ninety-degree angle. This embodiment of the present interlocking plate is useful, for example, when a fusion cage is placed at the L5/S1 location along the lower spine. The body of the L5 vertebrae extends downward at this location, making it difficult to insert screws into the vertebrae at a forty-five degree angle. An interlocking plate having an edge such as that shown in FIG. 14 allows insertion of screws into the L5 vertebrae at approximately a ninety-degree angle. The opposite edge of the interlocking plate in this embodiment of the invention is preferably similar to that shown in FIG. 5, thereby allowing an easier placement of screws within the body of the Si vertebrae. It is contemplated that yet another plate, having another asymmetrical arrangement of edges, may be used at another location along the spine, with the edges of the interlocking plate adapted to best fit the shape or angle of the vertebra at issue. Any suitable angle of combination of angles may be used with an interlocking plate in order to achieve the desired fit of the plate against vertebral bodies, and in order to achieve a desired angle of screw insertion therein.

In any of the embodiments of the present invention, upper and lower cage surfaces 12 and 14 may be provided with varying wedge configurations, so as to allow a desired degree of lordosis. A preferred angle of lordosis is approximately eight degrees, though various other angles may be desired for particular purposes, and any suitable method for determining desired angles may be used to construct an appropriate device failing within the scope of the present invention.

In any of the various embodiments of the present invention described above, upper and lower cage surfaces 12 and 14 may be provided in a variety of configurations, including but not limited to waffled, smooth, serrated, saw-toothed, or drilled configurations.

In any of the embodiments of the present invention, cage 10 may be used with or without interlocking plate 20. Likewise, interlocking plate 20 may be used with or without cage 10. It is preferred, however, that cage 10 and interlocking plate 20 are used together as described above.

In any of the embodiments of the present invention described above, fixation and fusion cage 10 may be packed with bone. This may be performed before the device is installed in a patient or after installation. In addition, a biologically active compound such as bone morphogenic protein may be included therein. In such embodiments, it is envisioned that interlocking plate 20 will act as a cap to contain the bone and/or biologically active material within cage 10.

The foregoing description of the embodiments of the invention has been presented for the purposes of illustration and description, and is not intended to be exhaustive or to limit the invention to the precise form disclosed. The description was selected to best explain the principles of the invention and practical application of these principles in order to enable others skilled in the art to best utilize the invention in various embodiments and with such modifications as are suited to the particular use contemplated. It is intended that the scope of the invention not be limited by the specification, but be defined by the claims as set forth below.

1. A fixation and fusion cage comprising:
an upper contoured surface;
a lower contoured surface; and
first and second side portions, said first and second side portions joining said upper and lower contoured surfaces such that said first and second contoured surfaces form opposing upper and lower surfaces of said fixation and fusion cage and said first and second side portions form opposing side walls of said fixation and fusion cage,
wherein an edge of at least one of said first contoured surface, said second contoured surface, said first side portion, or said second side portion is sized and shaped to form an interlocking portion for attachment of a plate portion thereto.

2. The fixation and fusion cage of claim 1, further comprising an interlocking plate portion having at least one mating portion adapted to engage said at least one interlocking portion such that said interlocking plate is rigidly attached thereto.

3. The fixation and fusion cage of claim 2 wherein said interlocking plate further comprises a central opening formed therein.

4. The fixation and fusion cage of claim 1 wherein each of said upper and lower contoured surfaces further comprise at least one opening formed therein.

5. The fixation and fusion cage of claim 1 wherein each of said first and second side portions further comprise at least one opening formed therein.

6. The fixation and fusion cage of claim 1 wherein said fixation and fusion cage is constructed from titanium, bone, carbon fiber, polyetheretherketone, nylon, polycarbonate, stainless steel, chromium-cobalt, or combinations thereof.

7. The fixation and fusion cage of claim 2 wherein said fixation and fusion cage is constructed from titanium, bone, carbon fiber, polyetheretherketone, nylon, polycarbonate, stainless steel, chromium-cobalt, or combinations thereof.

8. The fixation and fusion cage of claim 1 wherein said fixation and fusion cage is constructed from at least one radiolucent compound.

9. The fixation and fusion cage of claim 2 wherein said fixation and fusion cage is constructed from at least one radiolucent compound.

10. The fixation and fusion cage of claim 1 further comprising bone morphogenic protein provided in said interior space of said cage.

11. The fixation and fusion cage of claim 1 wherein a contour of at least one of said upper and lower contoured surfaces is derived from data obtained from multiple patient anatomies, said contour being shaped to mate with an adjacent vertebral surface during normal use of said fixation and fusion cage.

12. The fixation and fusion cage of claim 2 wherein said interlocking plate portion is sized and shaped such that when said interlocking plate portion is affixed to said cage and said cage is inserted into an intervertebral space, said cage is offset relative to the midline of the vertebral column.

13. A fixation and fusion cage comprising:
an upper contoured surface;
a lower contoured surface;
first and second side portions, said first and second side portions joining said upper and lower contoured surfaces such that said first and second contoured surfaces form opposing upper and lower surfaces of said fixation and fusion cage and said first and second side portions form opposing side walls of said fixation and fusion cage; and
at least one interlocking portion fixedly attached to an edge of at least one of said first contoured surface, said second contoured surface, said first side portion, or said second side portion, said interlocking portion sized and shaped for attachment of a plate portion thereto.

14. The fixation and fusion cage of claim 13, further comprising an interlocking plate portion having at least one mating portion adapted to engage said at least one interlocking portion such that said interlocking plate is rigidly attached thereto.

15. The fixation and fusion cage of claim 14 wherein said interlocking plate further comprises a central opening formed therein.

16. The fixation and fusion cage of claim 13 wherein each of said upper and lower contoured surfaces further comprise at least one opening formed therein.

17. The fixation and fusion cage of claim 13 wherein each of said first and second side portions further comprise at least one opening formed therein.

18. The fixation and fusion cage of claim 13 wherein said cage is constructed from at least one radiolucent material.

19. The fixation and fusion cage of claim 14 wherein said interlocking plate portion is sized and shaped such that when said interlocking plate portion is affixed to said cage and said cage is inserted into an intervertebral space, said cage is offset relative to the midline of the vertebral column.

20. A fixation and fusion cage comprising:
an upper surface;
a first side wall fixedly attached to said upper surface and extending away at an angle therefrom;
a lower surface fixedly attached to said first side wall and extending away at an angle therefrom;
a second side wall fixedly attached to said lower surface and to said upper surface; and
a plate portion,
wherein said upper surface, first side wall, lower surface, and second side wall define an interior space of said cage, and further wherein said cage comprises first and second end portions at opposing ends of a longitudinal axis thereof, and further wherein said plate portion is adapted to be removably engaged with either of said first or second end portions of said cage.

21. The fixation and fusion cage according to claim 20 wherein said fixation and fusion cage is constructed from at least one radiolucent material.

22. The fixation and fusion cage according to claim 20 wherein at least one of said upper and lower surfaces is shaped to mate with an adjacent vertebral surface during normal use of said fixation and fusion cage.

23. The fixation and fusion cage of claim 20 wherein said fusion cage is offset relative to the midline of the vertebral column when inserted into an intervertebral space.

24. The fixation and fusion cage of claim 1 wherein said plate portion comprises at least one opening adapted to receive a screw portion having a plurality of threads, and wherein an interior edge of said at least one opening is adapted to engage said plurality of threads.

25. The fixation and fusion cage of claim 20 wherein said plate portion comprises at least one opening adapted to receive a screw portion having a plurality of threads, and wherein an interior edge of said at least one opening is adapted to engage said plurality of threads.

26. A screw for attaching a medical implant to a bony portion of a patient's anatomy, said screw comprising:
a first plurality of threads, said plurality of threads sized and shaped to engage an interior surface of said bone portion; an
a second plurality of threads, said plurality of threads sized and shaped to engage an interior surface of an opening said medical implant.

27. The screw of claims 26 wherein said medical implant is a plate physically associated with a spinal fusion cage.

28. The screw of claim 26 further comprising a bullet tip portion for aligning said screw prior to inserting said screw in an opening in said bony portion.

29. The screw of claim 26 further comprising a tool-receiving portion sized and shaped to receive a tool therein for tightening or loosening said screw.

30. The screw of claim 26 wherein said screw is self-tapping.

31. The screw of claim 29 wherein said medical implant is a plate physically associated with a spinal fusion cage.

32. The screw of claim 30 wherein said medical implant is a plate physically associated with a spinal fusion cage.

33. The fixation and fusion cage of claim 20 wherein said plate portion comprises at least one opening for receiving a fastener therethrough, said fastener adapted to fasten said plate portion to a vertebrae of a patient.

34. The fixation and fusion cage of claim 33 wherein said at least one opening is positioned such that said fastener enters said vertebrae at about a forty-five degree angle relative to a longitudinal plane of said plate.

35. The fixation and fusion cage of claim 33 wherein said at least one opening is positioned such that said fastener enters said vertebrae at about a ninety-degree angle relative to a longitudinal plane of said plate.

36. The fixation and fusion cage of claim 33 wherein said plate portion further comprises:
a first opening for receiving a fastener therethrough, said first opening being positioned such that said fastener enters said vertebrae at about a forty-five degree angle; and
a second opening for receiving a fastener therethrough, said second opening being positioned such that said fastener enters said vertebrae at about a ninety degree angle.

37. The fixation and fusion cage of claim 33 wherein said plate portion further comprises:
a first edge, said first edge sized and shaped to snugly engage a first portion of at least one vertebrae of a patient receiving said fixation and fusion cage; and
a second edge, said second edge sized and shaped to snugly engage a second portion of at least one vertebrae of a patient receiving said fixation and fusion cage.
